(19) United States
(12) Patent Application Publication
(10) Pub. No.: US 2017/0333667 A1
(43) Pub. Date: Nov. 23, 2017

(54) TETHERLESS WEARABLE THERMAL DEVICES AND METHODS OF USING THEM FOR TREATMENT OF SLEEPING AND NEUROLOGICAL DISORDERS

(71) Applicant: Robert E. TUCKER, Sanibel, FL (US)

(72) Inventor: Robert E. TUCKER, Sanibel, FL (US)

(21) Appl. No.: 15/597,078

(22) Filed: May 16, 2017

Related U.S. Application Data
(60) Provisional application No. 62/337,721, filed on May 17, 2016.

(51) Int. Cl. A61M 21/02; A61B 5/0402; A61B 5/0476; A61B 5/11; A61B 5/00; A61B 5/0205; A61M 21/00; A61B 5/024; A61B 5/08

(52) U.S. Cl. CPC A61M 21/02; A61B 5/6803; A61B 5/0402; A61B 5/0476; A61B 5/0205; A61B 5/11; A61B 5/4809; A61B 5/4836; A61B 5/024; A61B 5/0816; A61B 2562/0219; A61M 2021/0066; A61M 2205/3368; A61M 2205/35; A61M 2205/3673; A61M 2210/0606; A61M 2205/8206

(57) ABSTRACT

Described herein are tetherless, forehead-mounted temperature regulation apparatuses configured to enhance sleep.

### CROSS REFERENCE TO RELATED APPLICATIONS

This patent application claims priority to U.S. provisional patent application No. 62/337,721, filed on May 17, 2016, and titled “TETHERLESS WEARABLE THERMAL DEVICES AND METHODS OF USING THEM FOR TREATMENT OF SLEEPING DISORDERS,” herein incorporated by reference in its entirety.

This patent may be related to one or more of: U.S. patent application Ser. No. 13/019,477, filed on Feb. 2, 2011 (now U.S. Pat. No. 8,425,583), titled “METHODS, DEVICES AND SYSTEMS FOR TREATING INSOMNIA BY INDUCING FRONTAL CEREBRAL HYPOTHERMIA;” U.S. patent application Ser. No. 13/868,015, filed on Apr. 22, 2013 (now U.S. Pat. No. 9,089,400), titled “METHODS, DEVICES AND SYSTEMS FOR TREATING INSOMNIA BY INDUCING FRONTAL CEREBRAL HYPOTHERMIA;” U.S. patent application Ser. No. 14/749,590, filed on Jun. 24, 2015, titled “METHODS, DEVICES AND SYSTEMS FOR TREATING INSOMNIA BY INDUCING FRONTAL CEREBRAL HYPOTHERMIA;” U.S. patent application Ser. No. 11/788,694, filed on Apr. 20, 2007 (now U.S. Pat. No. 8,236,038), titled “METHOD AND APPARATUS OF NONINVASIVE, REGIONAL BRAIN THERMAL STIMULI FOR THE TREATMENT OF NEUROLOGICAL DISORDERS,” U.S. patent application Ser. No. 14/938,705, filed on Nov. 11, 2015, titled “APPARATUS AND METHOD FOR MODULATING SLEEP;” U.S. patent application Ser. No. 12/288,417, filed on Oct. 20, 2008 (now U.S. Pat. No. 9,492,313), titled “METHOD AND APPARATUS OF NONINVASIVE, REGIONAL BRAIN THERMAL STIMULI FOR THE TREATMENT OF NERUOLOGICAL DISORDERS;” and U.S. patent application Ser. No. 14/341,642, filed on Jul. 25, 2014 (now U.S. Pat. No. 9,211,212), titled “APPARATUS AND METHOD FOR MODULATING SLEEP.” Each of these patents and patent applications is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The apparatuses and methods described herein may be used to improve sleep, including reducing sleep onset, improving sleep maintenance, increasing sleep duration, reduce awakenings, and increasing deep sleep relative to light sleep in a subject, including a subject suffering from a disorder that affects sleep such as insomnia. Thus, the apparatuses and methods described herein may be used to treat sleeping disorders such as insomnia.

### BACKGROUND

Sufficient and high-quality sleep is important for an individual's physical, mental, and emotional health. Despite various drugs and devices on the market for enhancing sleep and treating sleep disorders, disruption and irregularities, including insomnia, resulting in poor sleep is a widespread and pervasive problem. For example, previously described devices and techniques for the treatment of sleep disorders have included the use of cooling therapies, including applying cooling therapy to a patient's forehead, to enhance sleep. This has been described, for example, in U.S. Pat. No. 8,425,583, and U.S. Pat. No. 8,236,038, each of which are herein incorporated by reference in their entirety. Thus, there is evidence for enhancing sleep by cooling a subject's skin (e.g., forehead), perhaps by taking advantage of a mechanism involving cooling an underlying brain region. There is also recent work by these inventors suggesting that warming may also result in sleep enhancement, particularly warming relative to ambient temperature. The mechanism of action that temperature has on sleep has not been conclusively identified.

In general, studies have suggested that the control of sleep and thermoregulation (regulation of body temperature) are integrated at the level of the hypothalamus in the brain. Human studies have shown that manipulation of environmental temperature by various means can have an impact on sleep, however it is not well understood how selective regions of the body can influence hypothalamic sleep and thermoregulatory centers. Clinical insomnia and sleeplessness in general are characterized by transient or chronic difficulty initiating and maintaining sleep. It is unclear if alterations in thermoregulation play a significant role in the pathophysiology or treatment of insomnia or sleeplessness. Physiological and neuroanatomical studies show that the forehead is a region of the body that has unique properties and suggesting that it may play a prominent role in impacting the hypothalamic control of thermoregulation and, by extension, may influence hypothalamic thermoregulatory control of sleep.

Insomnia is often described as the inability to fall asleep easily, to stay asleep, or to experience quality sleep in an individual with adequate sleep opportunity. In the U.S., population-based estimates of either chronic (long-term) or transient (acute or short-term) insomnia range from 10% to 40% of the population, or 30 to 120 million adults. Similar prevalence estimates have been reported in Europe and Asia. Across studies, there are two age peaks for insomnia: 45-64 years of age and 85 years and older. Women are 1.3 to 2 times more likely to report trouble sleeping than men, as are those who are divorced or widowed, or have less education. In the U.S., the economic burden of insomnia approaches $100 billion in direct health care costs, functional impairment, increased risk of mental health problems, lost productivity, worker absenteeism, and excess health care utilization. Insomnia is recognized as a public health problem, contributing to more than twice the number of medical errors attributed to health care workers without insomnia episodes. Currently available treatments for insomnia are not satisfactory for a variety of reasons. Prescription drugs (e.g., sedative-hypnotics) that are given for insomnia treatment are associated with significant adverse events such as the potential for addiction/dependence, memory loss, confusional arousals, sleep walking, and problems with coordination that can lead to falls and hip fractures. The majority of insomnia patients prefer a non-pharmaceutical approach to treat their insomnia. Cognitive behavior therapy, while sometimes effective, is an expensive and labor intensive treatment that is not widely available and is not always covered by health insurance. Over-the-counter approaches to the treatment of insomnia include a variety of medications and devices that have not solved sleep problems, and inadequate clinical studies that fail to demonstrate significant effects in insomnia patients, as well as having potentially dangerous side effects. A large need exists, therefore, for a safe, effective, non-invasive treatment for treating sleep disorders and enhancing sleep.

Normal sleep cycles through different stages. The induction, maintenance and timing of wake, non-rapid eye movement sleep (NREM) or slow wave sleep (SWS), and rapid eye movement (REM) sleep stages are the result of complex interactions among multiple structures and mechanisms which are widely distributed throughout the brain. Reciprocal interactions between sleep and wake promoting systems ensure that the behavioral state of sleep-wakefulness is altered as required. Prominent among these sleep-promoting structures are the pontine tegmentum and adjacent neuronal groups in the brain that are involved in the generation of REM sleep features. On the other hand, NREM sleep is promoted by several areas in the brain, including the medial preoptic area (mPOA), the lateral preoptic area (lPOA), the ventrolateral preoptic area (vlPOA), the median preoptic nucleus (mnPO), and the medial septum, which are referred to as basal forebrain (BF) areas. External and internal factors influence the swing of sleep-wakefulness toward either sleep or awake state. The basal forebrain plays a role in integrating thermoregulation and sleep regulation.

Body temperature regulation (thermoregulation) is a fundamental homeostatic function that is regulated by the central nervous system. The preoptic area (or POA) of the hypothalamus is considered the most important thermoregulatory site in the brain on the basis of thermoregulatory studies, such as responses elicited by local warming and cooling, analysis of lesions, results from stimulation and single neuronal recording, and other techniques. Thermoregulation in the preoptic area is controlled by thermosensitive neurons. The thermosensitive neurons in the POA receive and integrate cutaneous (skin) and deep body thermal information. These neurons are tonically active at thermoneutral temperature, and control the thermoregulatory efferent pathway.

The concept of the POA as a sleep-promoting area and the posterior hypothalamus as a wake promoting area is supported by several lines of animal experiments employing stimulation, lesion studies, single unit recording, neural transplantation, functional magnetic resonance imaging (fMRI), and c-fos studies. The neural mechanism involved in the regulation of sleep and temperature and their interrelationship has been explored in various studies.

A thermoreceptor may be a peripheral thermoreceptor (e.g., a receptor on the skin or mucous membranes of a subject that monitors external temperature) or a central thermoreceptor (e.g., an internal receptor that monitors internal body temperature). The preoptic area of the hypothalamus contains temperature sensitive neurons (warm sensitive neurons (WSN) and cold sensitive neurons (CSN)). These neurons were identified in the preoptic area on the basis of in vivo and in vitro studies. Warm sensitive neurons are directly sensitive to (and fire in response to) locally warm temperatures and cold sensitive neurons are directly sensitive to (and fire in response to) local cool temperatures.

Several studies indicate that both ambient and body temperatures influence sleep architecture. The thermoregulatory pathway which initiates a heat or cold defense response in the body is conveyed by skin thermoreceptors, en route dorsal horn, and parabrachial nuclei, to the POA. Much attention has been given to the physiological role of the POA because of its ability to control both thermoregulation and sleep. Many of the observations cited earlier support the hypothesis that sleep is modulated by thermosensitive neurons of the POA. Although this relationship has drawn considerable interest, it is still not known whether there is a “cause and effect” relationship or whether these changes are merely coincidental. Notably, studies of skin temperature in insomnia patients have focused on distal skin temperatures in the feet and hands. Whether there are other more temperature sensitive regions of the body that can transmit temperature sensitive information to the POAH is not known.

Among body regions, the forehead has unique physiological and neuroanatomical properties that suggest it may play a prominent role in influencing the thermoregulatory hypothalamic modulation of sleep. The distribution of warm and cold spots has been shown to be highest over the face and forehead of all body parts Thermal sensation has been shown to be highest in the forehead of all body parts. Further, the forehead comprising glabrous (non-hairy) skin has been shown to play a prominent role in the body response to thermoregulation given that the heat transfer function and efficacy of glabrous skin is unique within the entire body based on the capacity for a very high rate of blood perfusion and the novel capability for dynamic regulation of blood flow. Despite extensive research, it is still not well understood how sleep is controlled, and sleep disorders including insomnia, remain a significant problem for a large number of people.

Described herein are methods and apparatuses capable of stimulating a response in a subject's body to improve sleep in the subject. These methods, systems and devices may stimulate temperature sensitive receptors (e.g., cold sensitive or warm sensitive neurons) in a subject's skin to enhance the subject's sleep. In particular, the apparatuses and methods described herein may use a tetherless (e.g., self-contained), wearable device (e.g., wearable on a forehead) apparatus that may be useful to decrease sleep onset (e.g., facilitate falling to sleep), decrease arousals, increase sleep duration, increase depth of sleep, and/or treat insomnia).

### SUMMARY OF THE DISCLOSURE

Described herein are apparatuses and methods, including methods of using the apparatuses, to enhance sleep. Enhancing sleep may include one or more of: reducing sleep onset latency, extending sleep duration, reducing awakenings, and/or increasing the duration of deeper sleep stages relative to stage 1 sleep in a subject (e.g., increasing the ratio of deeper sleep stage duration relative to stage 1 sleep duration). These apparatuses and methods may be applied to subjects (e.g., persons, patients, etc.) in need thereof; for example, the methods described herein may be used to treat a subject suffering from a sleeping disorder such as insomnia. In general, these apparatuses and methods apply (and may maintain) stimulation of temperature-sensitive receptors in a subject's body (skin) and through the receptor provide signals that enhances the subject's sleep. Any of the apparatuses and methods of using them described herein may be configured to apply heat and/or cooling to a subject's forehead using a self-contained (e.g., tetherless) apparatus.

For example, described herein are apparatuses for applying thermal energy to a subject's forehead to enhance sleep, the apparatus comprising: a conformable body configured to be worn on the subject's forehead having a skin-facing surface; a plurality of thermoelectric temperature regulators arranged across the skin-facing surface; a controller configured to apply power to cool the thermoelectric temperature regulators to between 1° C.-30° C.; and a holdfast configured to secure the apparatus to the subject's forehead.

The holdfast may be a strap, hat, headband, adhesive, or the like.

Any of these apparatuses may include one or more sensors. The sensor(s) may be configured to collect data from the subject and transit this data (wired or wirelessly) to the controller/processor in the apparatus, or a remote processor that is in communication with the controller in the wearable apparatus. The data may be used by the controller/processor to determine the sleep state of the subject wearing the device (e.g., awake, NREM (stage 1, 2 or 3), REM sleep, etc.), determine if the subject is wearing the apparatus and/or determine the parasympathetic status of the subject.

Modulation of the parasympathetic nervous system may be used to reduce sleep onset and maintain sleep. One manner in which the autonomic nervous system can be modulated is through the primitive autonomic nervous system reflex known as the diving reflex. The diving reflex is triggered by immersion of the body in cold water, and is characterized by a reduction in heart rate (HR) due to an increase in cardiac vagal activity, a primary efferent of the parasympathetic nervous system; this is often associated with vasoconstriction of selected vascular beds, due to increased sympathetic output to the periphery. Thus, any of the apparatuses and methods described herein may be configured to induce a diving reflex response in a patient wearing the apparatus by providing cooling to the forehead of the patient (including local, spot cooling). Sensors that may detect the parasympathetic state (e.g., heart rate, heart rate variability, etc.) may therefore also be used to modulate the applied cooling therapy and toggle between cooling and standby temperatures.

Thus, any of these apparatuses may include a sensor configured to detect one or more of the subject's autonomic state and the subject is sleep/wake state. The sensor may be an accelerometer integrated into the conformable body (e.g., detecting body position and/or motion, which may also be used to derive sleep state or simply sleep/awake status). The sensor may be configured to detect one or more of: body movement, respiratory rate, heart rate, electrocardiogram (ECG) signals, and electroencephalogram (EEG) signals.

In general, the controller is further configured to apply the power to cool the thermoelectric temperature regulator to between 1° C.-30° C. In some variations, the controller may control the applied temperature during an active cooling phase for a predetermined time period (e.g., 10 minutes to 60 minutes, 10 minute to 45 minutes, etc. or any time between 10 minutes and 3 hours, which may be user selected or automatically determined), or until the apparatus detects that the user is asleep. The apparatus may then toggle into a standby state and may regulate the temperature at a standby temperature (e.g., between 26° C. and 38° C., between 28° C. and 38° C., between 30° C. and 38° C., etc. or within a few degree, e.g., +/−2° C., of body surface/skin temperature). In some variations, the apparatus may apply the cooling in a ramp with decreasing temperature (e.g., decreasing from 30° C. or 25° C.) until the subject experiences a diving reflex, at which point the temperature may be sustained for a predetermined first treatment time period (e.g., 10 minutes to 60 minutes, 10 minute to 45 minutes, etc. or any time between 10 minutes and 3 hours).

For example, a controller may be further configured to apply the power to cool the thermoelectric temperature regulator to between 1° C.-30° C. when the subject is awake. The controller may be further configured to apply power to the thermoelectric temperature regulators to maintain a standby temperature of between 26-38° C. when the subject is asleep.

Any of the apparatuses described herein may be battery powered (and/or rechargeable). For example, the apparatus may further comprise a battery. Any of these apparatuses may include a charging circuit and/or a charging antenna configured to recharge the battery from power inductively received by the charging antenna. The charging antenna may be configured to power the controller and the thermoelectric temperature regulators when power is received by the charging antenna.

Any of these apparatuses may include a wireless communications circuit configured to wirelessly transmit and receive to and from the controller.

In general, the controller may be configured to apply energy in a pulsatile manner so that the thermoelectric temperature regulators apply variably cooling to the forehead (e.g., varying between the cooling temperature and a temperature that is slightly higher than the cooling temperature). For example, the energy may be applied in pulses having a pulse duration of less than 360 seconds.

Any of these apparatus may be configured for use with a cover, which may be removable or non-removable, having a thermally transmissive surface configured to be positioned over the skin-facing surface. For example, the cover may be a single-use cover to prevent dirtying of the device when worn and/or to help secure the device to the forehead.

Also described herein are apparatuses for applying thermal energy to a subject's forehead to enhance sleep that include: a conformable body configured to be worn on the subject's forehead having a skin-facing surface; a plurality of thermoelectric temperature regulators arranged across the skin-facing surface; a sensor; a controller configured to receive sensor data from the sensor and to determine when the subject is sleeping or awake; further wherein the controller is configured to apply power to cool the thermoelectric temperature regulators to a first temperature of between 1° C.-30° C. when the patient is awake and to apply power to cool the thermoelectric temperature regulators to a standby temperature of between 26-38° C. when the patient is asleep; and a holdfast configured to secure the apparatus to the subject's forehead.

Also described herein are methods of enhancing sleep using a tetherless thermally-regulated applicator, the method comprising: optionally attaching the tetherless thermally-regulated applicator to a subject's forehead; determining when the subject is sleeping or awake based on sensor data received from one or more sensors in communication with a controller in the tetherless thermally-regulated applicator; applying power to cool a plurality of thermoelectric temperature regulators on the tetherless thermally-regulated applicator to a first temperature of between 1° C.-30° C. when the patient is awake; and decreasing the power to the thermoelectric temperature regulators and maintaining the tetherless thermally-regulated applicator at a standby temperature of between 26° C.-38° C. when the patient is asleep.

Attaching may comprise attaching a holdfast to secure the tetherless thermally-regulated applicator to the subject's forehead.

Determining if the subject is asleep or awake may comprise receiving data from one or more sensors integrated into the tetherless thermally-regulated applicator. For example, determining if the subject is asleep or awake may comprise receiving data from one or more sensors wirelessly communicating with the controller of the tetherless thermally-regulated applicator, wherein the one or more sensors are separate from the tetherless thermally-regulated applicator. Determining if the subject is asleep or awake may include determining if the subject is asleep or awake based on sensor data received from one or more sensors configured to detect one or more of: body movement, respiratory rate, heart rate, electrocardiogram (ECG) signals, and electroencephalogram (EEG) signals.

Applying power to cool a plurality of thermoelectric temperature regulators on the tetherless thermally-regulated applicator to a first temperature may comprise applying power to the thermoelectric temperature regulators to cool to between 10° C.-25° C. when the patient is awake. Decreasing the power to the thermoelectric temperature regulators may comprise maintaining the tetherless thermally-regulated applicator at a standby temperature of between 32° C.-38° C. when the patient is asleep.

Also described herein are method of enhancing sleep using a tetherless thermally-regulated applicator attached to a subject's forehead, the method comprising: applying power, using a controller in the tetherless thermally-regulated applicator, to a plurality of thermoelectric temperature regulators on the tetherless thermally-regulated applicator to cool a thermal transfer surface on the tetherless thermally-regulated applicator to a first temperature of between 1° C.-25° C. for a therapy period and decreasing the power applied to the thermoelectric temperature regulators after the first duration to maintain the tetherless thermally-regulated applicator at a standby temperature of between 26° C.-38° C. for a standby period. Optionally, the method may include cycling between the therapy period and the standby period.

The therapy period may have a duration of greater than 10 minutes (e.g., greater than 15 minutes, greater than 20 minutes, greater than 30 minutes, greater than 40 minutes, greater than 45 minutes, greater than 1 hour, etc., including any value between 10 and 120 minutes).

The therapy period may end when either the patient falls asleep or after a predetermined time period. The controller may monitoring the sleep state, but if the patient has not fallen asleep by the end of the therapy period, the controller may switch to the standby period. The predetermined time period is greater than 10 minutes (e.g., greater than 15 minutes, greater than 20 minutes, greater than 30 minutes, greater than 40 minutes, greater than 45 minutes, greater than 1 hour, etc., including any value between 10 and 120 minutes).

The standby period may have a duration of greater than 10 minutes (e.g., greater than 15 minutes, greater than 20 minutes, greater than 30 minutes, greater than 40 minutes, greater than 45 minutes, greater than 1 hour, greater than 2 hours, greater than 3 hours, etc., including any value between 10 and 120 minutes). The standby period may end when the subject enters a new sleep state (e.g., awakens, transitions from NREM to REM, transitions within NREM from stage 1 to stage 2, stage 2 to stage 3, etc.).

Thus, in any of these variations, the method may also include determining, in a processor in communication with the controller, that the patient is asleep or awake, and transitioning between the therapy period and the standby period when the patient falls asleep.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a schematic illustration of the skin-contacting surface of one variation of the apparatus for applying thermal energy to a subject's forehead using a thermoelectric material to enhance sleep as described herein.

FIG. 1B is a schematic illustration of the opposite side of the apparatus of claim 1A.

FIGS. 2A-2C illustrate schematic partially transparent views through variations of apparatuses for applying thermal energy to a subject's forehead using a thermoelectric material to enhance sleep.

FIG. 3A is an exemplary block diagram illustrating an apparatus for applying thermal energy to a subject's forehead using a thermoelectric material to enhance sleep.

FIG. 3B is another exemplary block diagram illustrating an apparatus for applying thermal energy to a subject's forehead using a thermoelectric material to enhance sleep.

FIG. 4 is an example of an apparatus for applying thermal energy to a subject's forehead using a thermoelectric material to enhance sleep.

FIG. 5 illustrates a subject wearing an apparatus for applying thermal energy to a subject's forehead using a thermoelectric material to enhance sleep.

FIG. 6 is an example of an apparatus for applying thermal energy to a subject's forehead using a thermoelectric material to enhance sleep that may be batteryless and/or may be charged while sleeping using an inductive charger positioned beneath or near the sleeping subject.

FIG. 7 illustrates wireless communication between an apparatus for applying thermal energy to a subject's forehead using a thermoelectric material to enhance sleep, a remote processor (e.g., smartphone), and/or one or more sensors for monitoring patient parameters (including wearable sensors and/or bedding sensors).

FIG. 8A shows the inner (skin-facing) surface of an exemplary prototype of an apparatus for applying thermal energy to a subject's forehead using a thermoelectric temperature regulator (TTR), including eight TTR cooling surfaces arranged across the skin-facing side of the device, for contacting the skin (although a thermal spreader may be used to distribute the applied cooling/warming).

FIG. 8B shows the outer surface of the apparatus of FIG. 8A, showing the opposite side of the thermoelectric temperature regulators, including heat sinks.

### DETAILED DESCRIPTION

In general, described herein are apparatuses (e.g., a device and/or a system) for treating insomnia, or to generally improve and/or enhance healthy sleep even in non-insomniac subjects by applying a thermal therapy to the subject's forehead area. These devices typically include one, or more preferably a plurality of, e.g., two, three, four, five, six, seven, eight, nine, ten, etc. of thermoelectric temperature regulators, a controller applying power to regulate the temperature of the thermoelectric temperature regulators, a body portion, which may be flexible to conform to the subject's head, and a strap to hold the device on the subject's head. The apparatus may also include a power source, such as a battery or capacitive power source, etc. The apparatus may include one or more sensors, or it may be configured to wirelessly communicate with one or more sensors that provide information about the patient's sleep state (e.g., sleep stage), ECG, heart rate (e.g., heart rate variability), body movement, body/head position, body and/or ambient temperature, or the like.

Any of the apparatuses described herein may be specifically adapted for use with a sleeping subject. Thus, these apparatuses may be configured to operate without requiring a subject's input, including providing additional power to the device. As will be described in greater detail below, the controller may be configured to run the thermoelectric temperature regulators in a thermal profile that enhances sleep latency onset (the time to fall asleep) and/or sleep maintenance (sustaining sleep) in a manner that conserves the power.

Any of the apparatuses described herein may apply thermal therapy to the subject. Thermal therapy is defined as the application of a constant or varying temperature between 0° C. and 40° C. (e.g., between a lower temperature of 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C. and an upper temperature of 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., etc. where the lower range is always less than the upper range), for example between 10° C. and 15° C., between 10° C. and 20° C., etc. to all or part of the forehead area over the frontal cortex.

These apparatuses may be referred to as apparatuses for applying thermal energy to a subject's forehead using a thermoelectric temperature regulator comprising a thermoelectric material to enhance sleep. The thermoelectric temperature regulators material may be any appropriate thermoelectric material. Generally, a thermoelectric material may be given its ordinary meaning in the art and refers to materials in which a temperature change is generated at a surface of the material upon application of an electric potential (e.g., voltage and corresponding current), in accordance with the thermoelectric effect, often referred to by other names such as the Peltier, Thomson, and Seebeck effects. While a portion of the description herein describes thermoelectric materials, the present disclosure is not limited to thermoelectric materials, and other thermal adjustment apparatuses may be employed where appropriate. Non-limiting examples of suitable thermoelectric materials may include columns of p-type and n-type doped semiconductor materials, bismuth chalcogenides (e.g., Bi₂Te₃, Bi₂Se₃), lead selenide. Si—Ge alloys, skutterudites (e.g., including the formula LM₄X₁₂, wherein L is a rare earth metal, M is a transition metal, and X is a metalloid), or any other suitable thermoelectric materials.

The dimensions of the thermoelectric temperature regulators described herein may be appropriate so that the apparatus can both generate the appropriate temperature profile, may cover a sufficiently large region of the subject's forehead, and may remain flexible enough so that the apparatus, typically including an array of thermoelectric temperature regulators (also referred to herein as thermoelectric modules, thermoelectric elements or thermoelectric cooler/heaters), may conform comfortably to the subject's forehead. A thermoelectric temperature regulator may have any suitable thickness. For example, in some embodiments, the thickness may be selected such that the thermoelectric temperature regulator(s) may be comfortably held against the forehead. In some embodiments, the thickness of each of the thermoelectric materials may be between about 1 millimeter and about 5 millimeters (e.g., between about 1 millimeter and about 3 millimeters). Other thicknesses are also possible. Each of the thermoelectric materials, or modules that include the thermoelectric temperature regulator(s), may have a largest average cross-sectional dimension of between about 10 mm and about 4 cm (e.g., between about 30 mm and about 500 mm). Other average cross-sectional dimensions are also possible. Those skilled in the art would be capable of selecting an appropriate size for the thermoelectric material based upon the configuration of the device.

A thermoelectric temperature regulator may be provided in any suitable configuration. For example, a module may include thermoelectric materials sandwiched between ceramic plates, in some cases, for protection and support, as well as to provide thermal conductivity to the surface of the skin. The thermoelectric temperature regulator may be configured to be directly applied to the skin, or a thermally conductive intermediary material (e.g., a reusable or disposable cover, layer, etc.) may be interposed between the subject's skin and the surface of the thermoelectric temperature regulator. The material forming the outer skin-contacting surface of the apparatus may be an outer surface of the thermoelectric temperature regulator and may be washable. The outer surface may be a metallic (e.g., aluminum, stainless steel, etc.). The back (outward-facing) side of the apparatus in some variations may include one or more structures to help dissipate heat, including heat sinks, fans, or the like. Further, in some variations, the apparatus may be configured to include a soft material (e.g., a filling, such as a foam and/or cushioning material), which may also help distribute the waste heat, and may make the apparatus more comfortable for use while sleeping.

In general, the apparatuses described herein may not cover the eyes of the subject wearing them. Alternatively in some variations, an eye-covering portion may also be included.

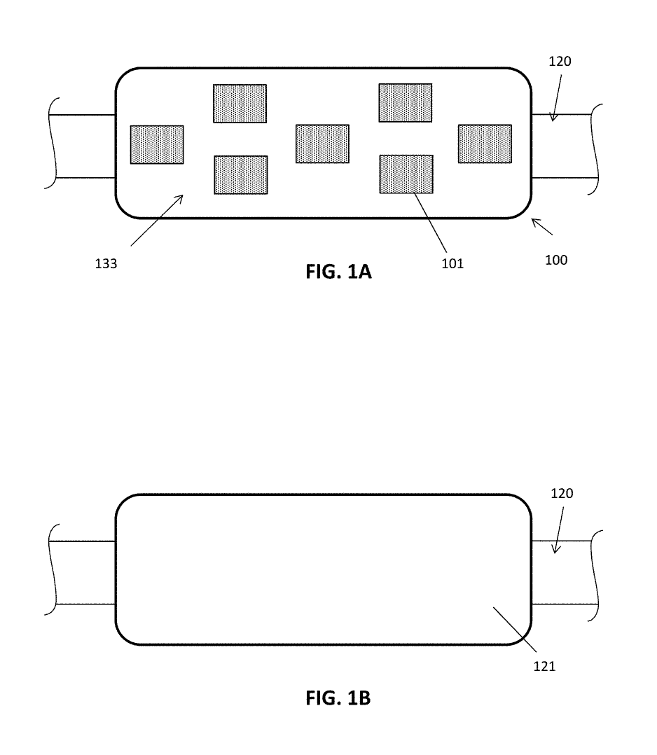

Any of these apparatuses may include a thermally insulative material positioned so as to surround and/or insulate the cooling portion of the thermoelectric temperature regulator (e.g., the skin-facing side, worn against the subject's skin) from the warming side of the thermoelectric temperature regulator. For example, an outward-facing surface of the apparatus maybe partially covered by an insulative material; regions coupled to the thermoelectric temperature regulator heating side may be coupled to a heat sink material to dissipate waste heat. Alternatively, all or a portion of the outward-facing surface of the device (see. e.g., FIG. 1B) may be exposed and/or may include a heat sink and/or fan. In certain embodiments, during use, the thermoelectric temperature regulators may be located between thermally insulative material and the surface of the skin. The thermally insulative material may be effective to retain the level of heat or cooling generated by the thermoelectric temperature regulator(s), adjacent the surface.

In general, the apparatuses described herein may be thin, lightweight wearable devices adapted to be worn on a subject's head while sleeping to provide cooling to the head. These apparatus may be flexible, and do not require a circulating cooling fluid.

As mentioned the devices may use one or more thermoelectric temperature regulators, which may be configured as thermoelectric coolers (e.g., TECs) that may be placed directly on the skin covering all or part of the forehead area. The use of a thermal spreader material can be used to help distribute the TEC temperature over a larger area than covered by the TECs. Thermal spreader materials are widely known in thermal management.

A conformable material may be used as an interface layer between the thermoelectric temperature regulators and the skin to improve comfort from the rigid thermoelectric temperature regulators surface. This conformable material may or may not act as a thermal spreader.

In some variations, the thermoelectric temperature regulators can be used in conjunction with known phase change materials or evaporative cooling to provide the desired temperature profiles.

Apparatuses and methods may be included to control the waste heat generated by the thermoelectric temperature regulators. As mentioned, one or more heat sinks and/or fans may be used. In addition, the apparatus may be adapted to provide evaporative cooling. For example, the apparatus may include a reservoir of an evaporative cooling material (e.g., water, alcohol, etc.) that may be released during use small amounts onto an evaporative cooling surface to increase heat transfer. This release may be temperature dependent. For example, the apparatus may include a temperature sensor to determine the temperature of the non-skin contacting side and may provide evaporative cooling by release of metered amounts of fluid material to allow evaporative cooling when the temperature is above a threshold temperature (e.g., greater than 40° C., greater than 45° C., greater than 50° C., etc.).

Any of the forehead-cooling apparatuses and methods described herein may include an evaporative heat sink for removing waste heat from the apparatus. For example, these apparatuses may include a heat sink and a wetting material disposed on the heat sink that is configured to hold a desired amount of cooling liquid (e.g., water), without adding an excessive heat transfer resistance or heat capacity. Alternatively or additionally, the apparatus may cycle the electrical power supplied to the thermoelectric temperature regulators. Thus, rather than hold the thermoelectric temperature regulators at a constant temperature, the apparatus may take advantage of the fact that the temperature receptors on the surface of the forehead, and particularly the cooling sensors, may respond to the change in the temperature. Thus, these apparatuses may provide efficient cooling to induce sleep onset and maintenance by cycling to a temperature (e.g., a low temperature of between 0° C. and 25° C., e.g., between 10° C. and 20° C., between 10° C. and 18° C., between 10° C. and 17° C., etc.) from a temperature that is slightly warmer (e.g., greater than 5° C. warmer, greater than 6° C. warmer, greater than 7° C. warmer, greater than 8° C. warmer, greater than 9° C. warmer, greater than 10° C. warmer, between 2° C. and 15° C. warmer, between 3° C. warmer and 14° C. warmer, between 4° C. warmer and 13° C. warmer, etc.).

Thus, any of these apparatuses may include a body having a skin-contacting surface configured to be worn flush against the surface of a subject's forehead that includes a plurality of thermoelectric temperature regulators (TEC modules), one or more heat sink thermally coupled to each of the thermoelectric temperature regulators, typically on the side opposite from the skin-facing side, to dissipate waste heat. In some variations the apparatus may also include a fan and/or a wetting material in thermal communication with the heat sink(s). Each of these apparatus may also include or be configured to communicate with a controller for controlling the temperature of the thermoelectric temperature regulators. The controller may also receive input from one or more sensor(s), including sensors for detecting the sleep state of the user, or for detecting if the user is experiencing a diving reflex, which may be associated with enhancing sleep by reducing sleep onset or sustaining sleep. The controller may also coordinate the cooling of the forehead using the apparatus by cycling the thermoelectric temperature regulators in accordance with a duty cycle, for example, a duty cycle greater than about 10% during an active cooling period. The controller may also switch from an active cooling period in which the forehead is cooled to the first target temperature of, e.g., between 10° and 25° C., or any other appropriate cooling range) and a standby temperature of between 26° C.-38° C., and/or a temperature close to the subject's normal skin temperature (which may be empirically determined or determined by sensing from the forehead or other regions). Switching between an active cooling temperature and a standby temperature may maintain the battery life while preventing sleep disruption.

In variations in which a wetting material is included to help remove (by evaporation) the excess/waste heat, the apparatus may include a refillable reservoir of fluid and/or a supply line connecting the wetting material and the reservoir. The reservoir may include a sponge or other porous material. The supply line may include a wicking material to convey a liquid from the reservoir to the wetting material; alternatively the reservoir (e.g., sponge) may be connected directly to the wetting material. The heat sink may be any appropriate thickness (e.g., from about 1 mm to about 15 mm). A binding layer may secure the wetting material to the heat sink (e.g., the wetting material may be disposed between the heat sink and the binding layer). The binding layer may be wrapped around an outer edge of the heat sink. Alternatively or additionally, the wetting material may be a sponge material that holds the fluid therein. The heat sink may include an etched surface and/or a contoured surface. The contoured surface may form a bend having a bend angle in a range from about 0 degrees to about 90 degrees. In one embodiment, the wetting material includes an antimicrobial agent. In another embodiment, the wetting material includes a hydrophilic material. A thickness of the wetting material may be from about 1 mm to about 3 mm. The wetting material may include tissue and/or cotton.

As will be described in greater detail herein, the apparatus may include a plurality of thermoelectric temperature regulators distributed along the inner surface of the apparatus. Although a thermally conductive ‘spreading’ material may be used to distribute the cooling from the thermoelectric temperature regulators along the skin-contracting side of the apparatus, in some variations the apparatus does not include an additional spreader, but instead applies spots of cooling at the desired locations. These spots may be discrete or diffuse.

In general, the controller for any of the apparatuses described herein may include control logic (hardware, firmware, and/or software) for controlling the application of energy to the thermoelectric temperature regulators (including feedback) to regulate the temperature of the apparatus. For example, control logic may be employed to power the TECs to reduce waste heat or reduce power consumption. The duty cycle of power applied to the TECs can be varied in various ways to reduce waste heat and reduce power consumption. In particular, any of the apparatuses described herein may be configured to apply pulsed cooling/heating, as will be described in greater detail below. Alternatively, stead-state heating and/or cooling may be applied.

The thermoelectric temperature regulators may be powered in any appropriate manner. The apparatus, including the thermoelectric temperature regulators, can be powered by a control unit placed on a table near the subject and power can be supplied by an electrical wire from the control unit to the TECs on the subject (e.g., via a wall power source or line). The apparatus, including the thermoelectric temperature regulators, could be powered by control unit that includes a battery located on the subject's body and wired to the TECs. The battery could be removed from the subject worn control unit and recharged in a remote charging station. Multiple batteries could be used whereby batteries could be charging while others are used to power the TECs and exchanged when needed.

In some variations, the apparatus may be batteryless or may include batteries that are charged during use. For example, the device may be powered and/or the batteries could be charged while in use by induction charging. For example a coil may be located on the subject and wired to the battery charging circuit and/or a charging coil may be included as part of the apparatus. A second induction charging coil for applying the power may be located in proximity to the first coil when the apparatus is in use and/or charging, to induce induction charging of the system. As mentioned, the induction charging could provide all of the power to the TECs without the need for batteries. For example, an induction coil may be included in the bedding (e.g., mattress, pillow, etc.), headboard, bedframe, nightstand, wall-mount, etc. and positioned so that the apparatus may receive power when the subject is wearing the apparatus and lying in the bed.

The control unit may control power to the thermoelectric temperature regulators by any number of commonly known means such as pulse width modulation or simple analog circuits. The temperature of the thermoelectric temperature regulators and or the skin temperature and or the interface material can be monitored by one or more sensors and controlled by a microprocessor and software or it could be controlled by analog circuits. In one variation the power supplied to the thermoelectric temperature regulators is controlled by a time dependent algorithm. In one variation the power supplied to the thermoelectric temperature regulators is controlled by a bio feedback algorithm such as sleep staging information gathered by various sensors/software commonly used to determine sleep staging. In one variation, the battery power could be conserved by reducing the power to the thermoelectric temperature regulators when sensors indicate the subject is in stage 1 or stage 2 sleep and could be increased when it is detected the subject is no longer in the desired sleep stage. Other measurements, such as body movement or EEG signals may be used for TEC control.

FIG. 1 is a schematic illustration of one example of an apparatus 100 as described herein configured as a forehead cooling and/or heating. The apparatus 100 including a plurality of thermoelectric temperature regulators 101 thereon. In some variations fewer (e.g., just 1, just 2, etc.) or more (e.g., 8, 9, 10, etc.) may be used and may be positioned thereon in any arrangement, including arrangements specifically configured to preferentially modulate a parasympathetic and/or sympathetic response. The thermoelectric temperature regulators are shown as rectangular, however, they may be any shape, including round, oval, square, interlocking, etc. In FIG. 1, the body of the apparatus is shown as rectangular, however, it may be any shape, and in particular, may be a shape that is configured specifically for placement on the forehead. The body may be a conformable body 133 configured to flex, bend, and/or otherwise conform to the curvature of the subject's forehead. The thermoelectric temperature regulators may be arranged along the body so that they contact the non-hairy skin of the forehead on most (e.g., average) subjects. The body of the device may be shaped as a curved or curveable shape to match the curvature of the forehead. The apparatus may include a strap 120 or other securement to hold the device over the subject's forehead.

In FIG. 1A the apparatus has 7 thermoelectric temperature regulators arranged across the surface. The thermoelectric temperature regulators are separated by space, and the thermoelectric temperature regulators may be positioned on a substrate so that the body can be flexibly positioned over the forehead.

As mentioned above, the thermoelectric temperature regulators, during use, may be configured to be positioned directly adjacent to the surface of the user's skin. The apparatus may include multiple thermoelectric temperature regulators positioned at the surface of the skin, and may optionally include a thermally conductive material (e.g., heat sink) 121 located on the opposite side of the apparatus (e.g., the outward-facing side when worn), as shown in FIG. 1B, in a manner that covers the thermoelectric temperature regulators. A thermally conductive material may dissipate heat to and/or from the thermoelectric temperature regulator(s), as desired. The thermally conductive material may include any suitable material, such as metal (e.g., aluminum, copper, stainless steel, etc.), thermally conductive polymer, porous ceramic, or another appropriate material. In some variations, rather than a thermally conductive material, a thermally insulative material may be located on the side of the thermoelectric temperature regulator opposite the skin, covering the thermoelectric temperature regulator(s). Covering the thermoelectric temperature regulator(s) with a thermally insulative material may enhance the effects of thermal pulsing at the surface of the skin, when pulsing is used to apply cooling or heat. It is not required for the thermoelectric temperature regulators to be covered by a thermally conductive or insulative material. For example, a thermal dissipation apparatus may be spaced from or located adjacent to the thermoelectric temperature regulators, without covering the thermoelectric temperature regulators. Alternatively or additionally, the thermally conductive or insulative material may be arranged so as to cover a portion of the thermoelectric temperature regulators.

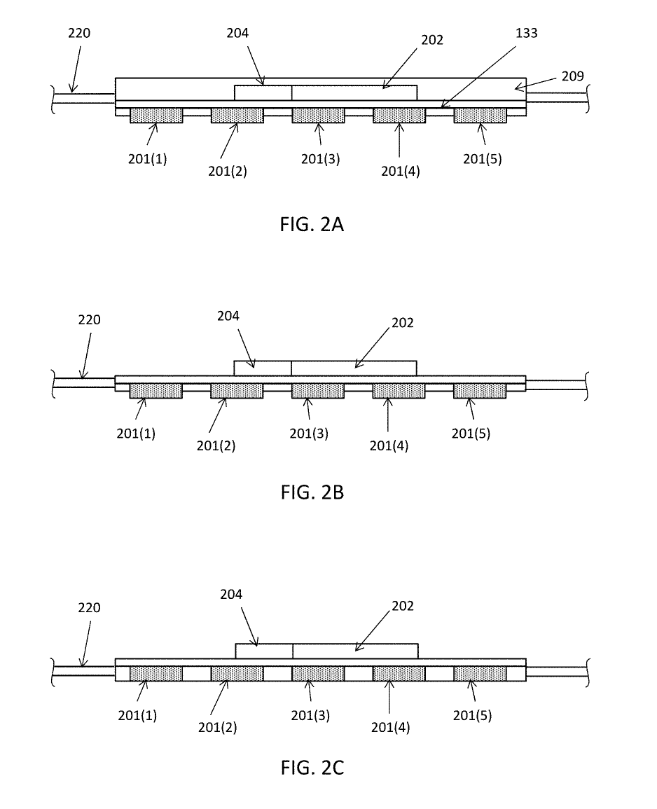

The apparatus may be connected to a power source 204 (e.g., battery, plug-in outlet, etc.) and a controller 202, for applying appropriate signals to the thermoelectric, for manipulating the temperature at the surface of the skin. For example, FIGS. 2A-2C illustrate variations of apparatuses shown in side, partially transparent views, so that arrangement of parts is visible.

In FIG. 2A, the apparatus includes a plurality of thermoelectric temperature regulators 201(1), 201(2), 201(3), 201(4), 201(5), that are arranged along the bottom, skin-facing side of the apparatus. In this example the bottom of the thermoelectric temperature regulators may be configured to contact the skin of the forehead; alternatively a cover that is thermally transmissive or that includes a thermally transmissive window, may be worn over the apparatus so that the thermoelectric temperature regulators can contact the skin. As shown in FIG. 2A, the thermoelectric temperature regulators project slightly from the bottom of the apparatus, but are arranged in a substrate 215, 217. The substrate may be flexible and may include electrical connections between the thermoelectric temperature regulators, the controller 202 and the power supply 204. The connectors may be wires or traces, and they may be formed on or in the flexible substrate(s). In FIG. 2A, the back side of the thermoelectric temperature regulators, facing away from the patient when worn, are shown covered in a material, such as a thermally conductive material as discussed above. This may protect the apparatus while allowing rejection of unwanted heat, e.g., via a heat sink.

The controller may have one or more inputs and/or outputs to accommodate user control of the device in a suitable and convenient manner. For example, the controller may be controlled by an ‘on’ button, an ‘off’ button, a timer, a mode set, etc.

FIG. 2B shows an example of an apparatus similar to that shown in FIG. 2A. In this example, the apparatus is nearly the same as shown in FIG. 2A, but does not include the (optional) covering 209 shown in FIG. 2A. The conformable body 133 of the device may be pre-shaped to fit a generic forehead (e.g., having a concave shape) and/or may be flat and configured to bend and conform. Any of these apparatuses may include cuts, slits, hinged regions or other structures to enhance bending or conforming to the head (forehead) shape. In any of these apparatuses the body may be formed at least in part of a flexible and/or stretchable fabric or other material. Similarly, FIG. 2C is another example in which the apparatus does not include a cover as shown in FIG. 2A. In this example, the thermoelectric temperature regulators are embedded and flush with the substrate. This may allow better surface contact between the target, e.g., skin, material. Additional elements, not visible in the parent application may also be included, such as one or more sensors (e.g., temperature sensors, accelerometers, etc.), an inductive charging antenna (e.g., coil), charging circuitry, a clock, a display and/or indicator lights, etc.

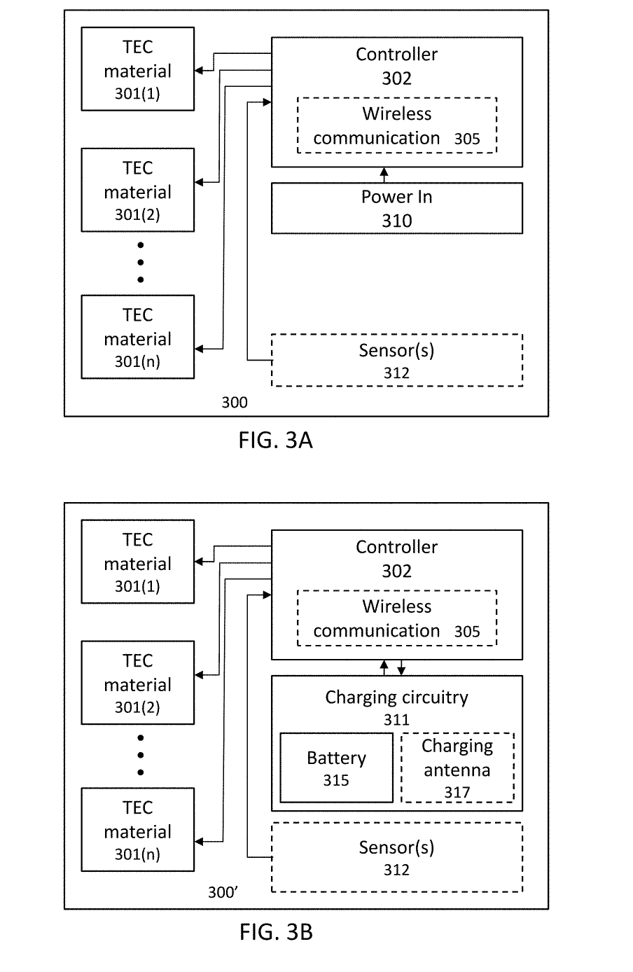

For example, FIG. 3A-3B schematically illustrate examples of apparatuses 300. In FIG. 3A, the apparatus includes a plurality of n thermoelectric temperature regulators 301(1), 301(2), . . . 301(n), each of which are controlled by a controller 302. The controller may communicate with (e.g., transmit to, receive from) a remote processor, such as a smartphone, laptop, computer, or dedicated control device. In FIG. 3A an optional wireless communication sub-system 305 may be included. This sub-system (which may be integrated with and/or part of the controller 302) may include a communications antenna, e.g., for RF, near field, Zigbee, Bluetooth (near field Bluetooth), etc. In some variations the apparatus may also include one or more sensors 312. The sensors may be biological/physiological sensors, such as temperature sensors, oxygenation sensors, ECG sensors, galvanic skin response sensors, etc. The sensors may be integrated into the device, or they may be separately coupled to the user (e.g., worn as part of a bandage, etc.).

The apparatus (system) of FIG. 3B is similar to what is shown in FIG. 3A, but also includes charging circuitry 311. As mentioned, the applicator may be charged inductively either before, during or after use. As mentioned, in some variations the charging circuitry may also be used for communications, e.g., for telemetry, and/or may be integrated into the controller circuitry 302.

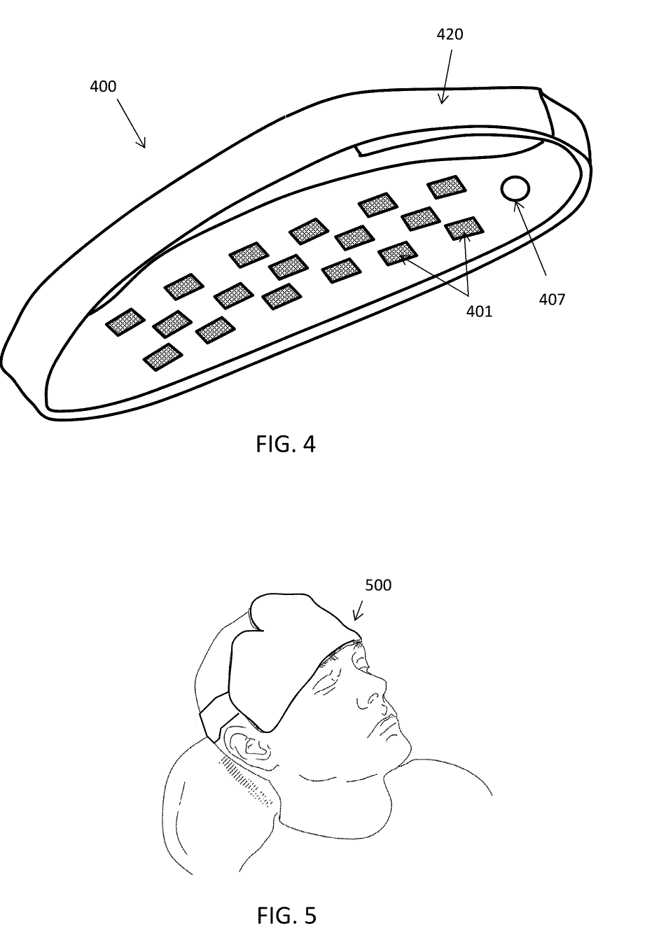

FIG. 4 is an example of an apparatus 400 shown as integrated with a holdfast 420, shown in FIG. 4 as a strap, for securing the device to the head and over the forehead, as shown in FIG. 5. The holdfast may also or alternatively be an adhesive (e.g., releasable skin adhesive), cap, headband, or the like. Returning to FIG. 4, the apparatus includes a plurality of small and thin thermoelectric temperature regulators 401 arranged along the internal skin-contacting surface. Any appropriate strap may be used, and it may be adjustable to fit different sizes of heads. The strap in FIG. 4 may be secured by Velcro and/or one or more attachments (e.g., snaps, buttons, buckles, etc.) to the head. In FIG. 5, a subject is shown wearing an apparatus 500 and lying down on a bed.

Any of these apparatuses may include one or more sensors 4007 which may be integrated into the forehead applicator 400 or separate from it. The apparatus may also include the controller (e.g. one or more processors, not visible in FIGS. 4 and 5) and one or more batteries, including rechargeable batteries, as discussed above.

All of the elements of the apparatuses described herein, i.e., the thermoelectric temperature regulators, thermally conductive material, power source and controller, may be suitably held together by an appropriate band. The band may be flexibly adjustable so as to allow for the thermoelectric temperature regulators to be comfortably and suitably positioned against or otherwise adjacent the surface of the skin such that thermal energy (e.g., thermal pulses) generated by the thermoelectric temperature regulators are effective to provide the subject with a preferred thermal sensation (cooling to between 0° C.-25° C., etc.). For some embodiments, the band may exhibit relatively rigid mechanical behavior, providing support for the overall device. It can be appreciated that the band or strap may have any suitable structure and, in some cases, may have stylistic aspects which may lend the device to be worn over the head and face (e.g., forehead) while sleeping or preparing for sleep. The strap may include any suitable material, such as, but not limited to, metal, plastic, rubber, leather, synthetic leather, or combinations thereof.

As mentioned, the thermoelectric temperature regulators may be positioned directly adjacent to a surface of the user's skin, in accordance with aspects of the present disclosure, the thermoelectric temperature regulator(s) are not required to be in direct contact with the user's skin; for example, an additional layer (not shown in the figures) may be placed between the thermoelectric temperature regulator(s) and the surface of the skin. For example, a thermally conductive or insulative layer, a protective layer, a support layer (e.g., for added comfort), or another appropriate material.

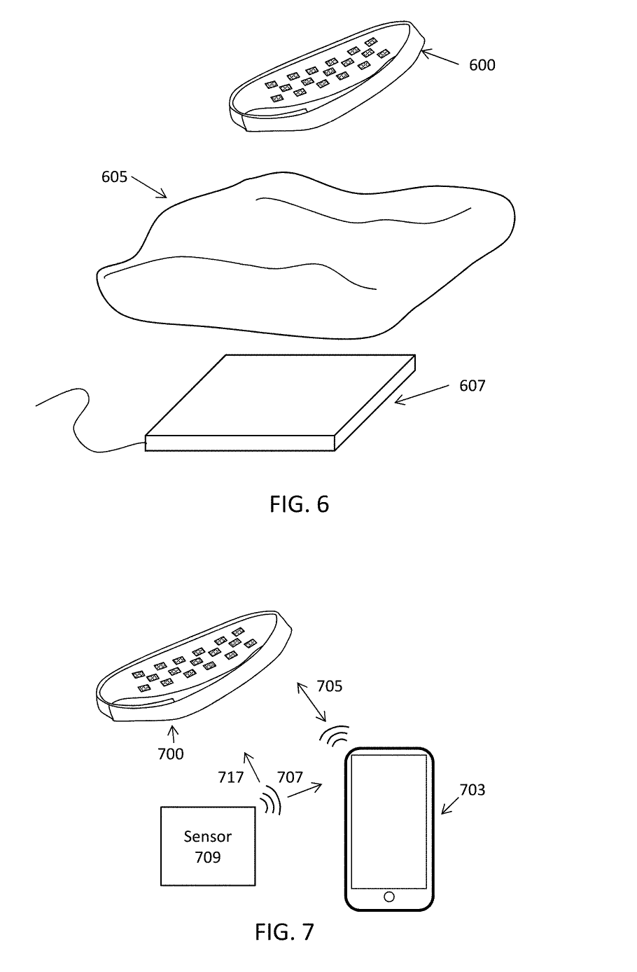

FIG. 6 illustrates an apparatus configured as a system that includes any of the forehead-wearable applicators 600 having a plurality of thermoelectric temperature regulators described above, which includes an antenna for receiving power from a charging pad 607. The charging pad may be integrated into the bedding (e.g., pillow 605, mattress, etc.), bed, headboard, etc. or it may be placed near the patient's head so that the forehead-wearable applicator may charge an apparatus and/or provide power directly so that it may be used without a battery.

FIG. 7 illustrates another example of an apparatus 700 (configured as a system) that also communicates with a separate, external to the apparatus, computing device, such as a smartphone 703 or the like. The smartphone may receive monitoring information and may store it and/or process it. In FIG. 7, the apparatus may also include one or more sensors 709. The sensors may be integrated into the applicator 700, or they may be separate, as shown in FIG. 7. Alternatively or additionally, the sensors may be body-worn sensors or sensors present in the subject's garments, bedding (sheets, mattress, pillow, sleepwear, etc.). Multiple sensors may be used and may provide this information to the controller of the apparatus.

In general, the controller may include a processor that may determine when and how much energy to apply to the thermoelectric temperature regulators to apply cooling or heating to the forehead through the device. In some variations, as shown in FIG. 7, the apparatus may operate in conjunction with a remote processor such as a smartphone 703. Thus, in some variations the processor of the smartphone may be used to determine the timing and/or energy applied to the thermoelectric temperature regulators.

In operation, the apparatus may be configured to apply continuous energy or intermittent energy to the thermoelectric temperature regulators. Alternatively or additionally the apparatus may be configured to generate a series of thermal pulses in succession by applying pulsatile energy to the apparatus. This thermal pulsing may result in an enhanced thermal sensation for a user which may require lower energy than constantly applied power, and yet may activate the thermal neural receptors (e.g., cold receptors) on the forehead comparable to continuously applied temperature.

A thermal pulse may include a transient, reversible temperature change of the thermoelectric temperature regulator, where the temperature changes from an initial temperature to another temperature, quickly followed by a return temperature change back to the initial temperature, or a temperature close to the initial temperature. The time course may be between, for example, 1 second and 360 second (e.g., 1 second and 120 seconds, etc.). A thermal pulse may include a first temperature adjustment at a surface from a first temperature to a second temperature (e.g., at an average rate of 0.1°-10.0° C./sec), and a second temperature adjustment at the surface from the second temperature to a third temperature (e.g., also at an average rate of 0.1°-10.0° C./sec). In such a thermal pulse, the difference in magnitude between the first temperature and the third temperature may be less than 25% of the difference in magnitude between the first temperature and the second temperature. Further, in some cases, the magnitude of the first average rate may be greater than the magnitude of the second average rate.

The pulsatile stimulation described herein may result in a comparable effect compared to steady-state, e.g., constant applied temperature and/or electrical signal modes, so as to maintain long-time scale applications of heating or cooling. For example, thermal pulses may result in continuous thermal stimulation for the human skin. Varying the temperature at the surface of the skin by generating thermal pulses may give rise to a heating or cooling effect that is perceived by the individual that are greater than or equal to those perceived at steady state.

Any of the apparatuses described herein may also be configured to apply energy to maintain the apparatus in a neutral or standby mode in which the apparatus is cooled and/or heated to a neutral temperature (e.g., skin temperature) when not actively cooling/heating, to prevent discomfort when sleeping. For example, the apparatus may be actively used prior to sleeping by maintaining the temperature, e.g., a therapeutic temperature of between 0° C.-25° C. (e.g., between 10° C. and 15° C., etc.). Once the subject wearing the apparatus on their forehead falls asleep, the apparatus may, detecting that the subject is asleep, adjust the temperature to a neutral temperature (e.g., 36° C.-37° C.) while the patient remains asleep. This neutral temperature may keep the device, which may otherwise insulate the head and lead to discomfort, from potentially rousing the patient from sleep. Using a sleep-activated neutral mode as described herein may also help conserve battery power.

### EXAMPLE

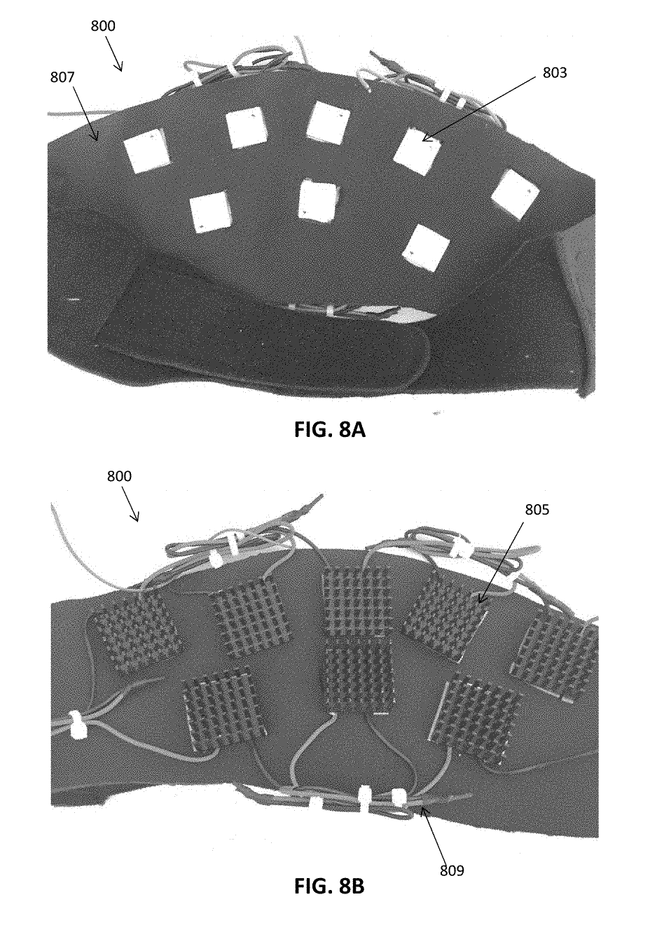

FIGS. 8A-8B illustrate an example of an apparatus for enhancing sleep onset and/or maintaining sleep and/or treating a sleep-related (e.g., neurological) disorder. In this example, a prototype forehead worn apparatus was built having a flexible, wearable substrate 807 that is thermally insulative, and through which a plurality of thermoelectric temperature regulators 803 are attached. The cooling side 803 of the thermoelectric temperature regulators are on the skin-facing side, while the warming side 805 are on the opposite side of the apparatus, as shown in FIG. 8B, configured to be worn facing away from the forehead.

In FIG. 8A, eight 15 mm by 15 mm thermoelectric temperature regulators are shown attached at discrete location for contacting the surface of a patient's skin. Each thermoelectric temperature regulators is connected (in the prototype wires 809 are visible, though they may be integrated into the fabric and/or covered. The thermoelectric temperature regulators may provide up to 8 W of cooling (e.g., between 2-8 W, between 3-7 W, between 4-8 W, etc.) using, for example, a power source such as a battery (e.g., Li battery, such as a 0.75″ by 2.5″ inch battery) that may be rechargeable.

In FIG. 8A, the device was tested and shown to be able to cool a subject's forehead over three hours of near-continuous use. The apparatus controller (not visible in FIGS. 8A-8B) may be included in the apparatus and may regulate the power to the apparatus. In FIG. 8B, the opposite side of the apparatus, which is worn so that it faces outward, shows the exposed backs of the thermoelectric temperature regulators to which a heat sink 805 has been attached. In practice, the battery or batteries (not shown in FIGS. 8A-8B) may be mounted on the wearable apparatus along with the control circuitry. The entire apparatus may be worn against the skin of the forehead, as shown in FIG. 5, above. The example devices shown above include a strap 813 holding the conformable temperature-regulated cooling surface of the thermoelectric temperature regulators against the skin; however, in some variations no strap is needed. The device may be adhesive held to the forehead surface, or other attachments may be used.

In any of the methods and apparatuses described herein, the apparatus may adjust the treatment (e.g., the temperature and/or timing) according to the patient's sleep cycle. Alternatively or additionally, any of the methods and apparatuses described herein may adjust the treatment based on the state of the subject's autonomic nervous system (e.g., sympathetic to parasympathetic ratio) and/or based on the response of the subject's parasympathetic and/or sympathetic nervous system. For example, these methods and apparatuses may include one or more sensors for measuring an indicator of the patient's autonomic nervous system response; this sensor data may be interpreted by the controller/processor, and may be used to adjust one or more of the temperature and/or timing of the therapy applied by the applicator to the subject's head (e.g., feedback).

Any appropriate sensed data for determining sleep stage (awake, NREM, REM, etc.) and/or determining the state of the autonomic nervous system (e.g., parasympathetic/sympathetic) may be used. For example, ECG, EEG, heart rate, heart rate variability, blood pressure, galvanic skin response, and/or any other indicator known to monitor autonomic function, an in particular parasympathetic function may be used.

It may be particularly helpful, but not necessary, to use one or more sensors configured to detect when a subject wearing the apparatus is experiencing a diving reflex. As mentioned, the apparatus may use feedback to adjust the temperature of the device and/or to switch between an active cooling (e.g., in a first range of between 0° C. and 25° C., or other cooling range) and a standby temperature (e.g., of between 26° C. and 38° C., e.g., between 30° C. and 38° C., between 32° C. and 38° C., between 34° C. and 38° C., etc.).

For example, a diving reflex may be detected by detecting peripheral vasoconstriction, slowed pulse rate, redirection of blood to the vital organs to conserve oxygen, release of red blood cells stored in the spleen, and heart rhythm irregularities. One or more sensors that detect and/or characterize a subject's diving reflex may be used in any of the methods an apparatuses describe herein. For example, the diving reflex may typically cause a change in heart rate of between 5-35% (e.g., 10-25%) within a few minutes (e.g., within 5 minutes, within 4 minutes, within 3 minutes, within 2 minutes, within 60 seconds, within 55 seconds, within 50 seconds, within 45 seconds, within 40 seconds, within 35 seconds, within 30 seconds, within 25 seconds, within 20 seconds, within 15 seconds, within 10 seconds, etc.). Thus, any of the apparatuses described herein may include a sensor configured to detect heat rate; this sensor(s) may be present on the applicator, or the sensor(s) may be separate from the applicator but in communication with the processor of the apparatus. For example, the subject may wear a wearable sensor that communicates with the apparatus. Sensors for detecting hear rate may include electrical (e.g., ECG) sensors, optical sensors (e.g., pulse oximetry sensors), vibration/motion sensors (e.g., accelerometers), etc. Alternatively or additionally, one or more sensors for detecting peripheral vasoconstriction may be used, and may be integrated into the apparatus or may communicate with the apparatus (e.g., pulse oximetry from one or preferably more locations, such as the hand/arm/finger and forehead). Changes in red blood cell levels may also be noninvasively detected and used to detect the presence and/or magnitude of a diving reflex.

One or more sensors may be included as part of the apparatus, including as part of the forehead applicator, as shown, or they may be separate from the applicator. As mentioned, these sensors may be for detecting one or more of: heart rate, heart rate variability, blood pressure, electroencephalogram, electrocardiogram, galvanic skin response, etc. The sensors may provide data to the processor/controller of the apparatus, where this data may be interpreted to determine the parasympathetic response or status of the patient. For example, the apparatus may be configured to determine if the patient is experiencing a diving reflex, or how robust a diving reflex the patient is experiencing, and may adjust the timing and temperature accordingly.

For example, any of the methods and apparatuses described herein may be configured to adjust temperature and/or timing of the apparatus based on the EEG, HRV and/or other sleep monitoring techniques, by themselves or in conjunction with an indicator of the diving reflex. The apparatus may vary the temperature applied throughout the sleep period based on feedback signals including feedback reflecting the sleep state or stage (e.g., awake, NREM (stage 1, stage 2, stage 3), REM etc.) and the diving reflex. In some variation the apparatus may adjust the temperature of the applicator in order to achieve and maintain a diving reflex in the subject, as determined by one or more sensors.

In one example, the temperature of the applicator may be controlled based on the heart rate. For example, the processor may monitor the heart rate to identify a change from an initial heart rate to a drop of more than 10% (e.g., between 10-35%) from the initial heart rate within a predetermined time period (e.g., 5 minutes, 4 minute, 3 minutes, 2 minutes, 1 minute, etc.), which may indicate the diving reflex. In a subject that is not yet asleep, the applicator may be cooled to a temperature that is ramped down (e.g., from body temperature, e.g., 37° C., or room temperature) gradually until the diving reflex is detected. Upon detection of the diving reflex (using one or more indicator, such as HR, HRV, blood pressure, vasoconstriction, rise in red blood cells, etc.) the temperature may be held steady. This procedure may therefore allow the cooling temperature to be customized to each patient/subject and for an individual subject between sessions, as some patients may respond to a much lower or higher temperature to the induction of the diving reflex.

Detection of the diving reflex may also or alternatively be used to start a timing for the application of the temperature regulation of the therapy. For example, the temperature of the apparatus may be held at or below the temperature at which a diving reflex response is determined for a predetermined maintenance time period (e.g., 10 minutes, 15 minute, 20 minutes, 25 minutes, 30 minutes, 35 minutes, etc.) and then increased to a second (e.g., standby) temperature for a second (e.g., standby) predetermined time period. The apparatus or method may then cycle one or more time through cooler temperatures (e.g., temperatures inducing a diving reflex, which may be the same as the first iteration or may be determined by monitoring the patient) and standby temperatures. In some variations, the temperature may be adjusted within a cycle, for example, in order to maintain the subject at the diving reflex.

In general, a processor/controller of the apparatus may receive the data from the one or more sensor(s) and may analyze and interpret the data. As mentioned above, the processor may be part of the apparatus or it may, in some variations, be separate (e.g., remote) from the apparatus, such as a smart phone processor to which the apparatus communicates.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An apparatus for applying thermal energy to a subject's forehead to enhance sleep, the apparatus comprising:
a conformable body configured to be worn on the subject's forehead having a skin-facing surface;
a plurality of thermoelectric temperature regulators arranged across the skin-facing surface;
a controller configured to apply power to cool the thermoelectric temperature regulators to between 1° C.-30° C.; and
a holdfast configured to secure the apparatus to the subject's forehead.

2. The apparatus of claim 1, further comprising a sensor configured to detect one or more of the subject's autonomic state and the subject's sleep/wake state.

3. The apparatus of claim 2, wherein the sensor is an accelerometer integrated into the conformable body.

4. The apparatus of claim 2, wherein the sensor is configured to detect one or more of: body movement, respiratory rate, heart rate, electrocardiogram (ECG) signals, and electroencephalogram (EEG) signals.

5. The apparatus of claim 2, wherein the controller is further configured to apply the power to cool the thermoelectric temperature regulator to between 1° C.-30° C. until the subject experiences a diving reflex.

6. The apparatus of claim 2, wherein the controller is further configured to apply the power to cool the thermoelectric temperature regulator to between 1° C.-30° C. when the subject is awake.

7. The apparatus of claim 6, wherein the controller is further configured to apply power to the thermoelectric temperature regulators to maintain a standby temperature of between 26-38° C. when the subject is asleep.

8. The apparatus of claim 1, further comprising a battery.

9. The apparatus of claim 7, further comprising a charging circuit and a charging antenna configured to recharge the battery from power inductively received by the charging antenna.

10. The apparatus of claim 1, further comprising a charging antenna configured to power the controller and the thermoelectric temperature regulators when power is received by the charging antenna.

11. The apparatus of claim 1, further comprising a wireless communications circuit configured to wirelessly transmit and receive to and from the controller.

12. The apparatus of claim 1, wherein the controller is configured to apply energy in a pulsatile manner so that the thermoelectric temperature regulators apply pulsed cooling to the forehead.

13. The apparatus of claim 12, wherein the energy is applied in pulses having a pulse duration of less than 360 seconds.

14. The apparatus of claim 1, further comprising a cover having a thermally transmissive surface configured to be positioned over the skin-facing surface.

15. An apparatus for applying thermal energy to a subject's forehead to enhance sleep, the apparatus comprising:
a conformable body configured to be worn on the subject's forehead having a skin-facing surface;
a plurality of thermoelectric temperature regulators arranged across the skin-facing surface;
a sensor;
a controller configured to receive sensor data from the sensor and to determine when the subject is sleeping or awake; further wherein the controller is configured to apply power to cool the thermoelectric temperature regulators to a first temperature of between 1° C.-30° C. when the patient is awake and to apply power to cool the thermoelectric temperature regulators to a standby temperature of between 26-38° C. when the patient is asleep; and
a holdfast configured to secure the apparatus to the subject's forehead.

16. The apparatus of claim 15, wherein the sensor is an accelerometer integrated into the conformable body.

17. The apparatus of claim 15, wherein the sensor is configured to detect one or more of: body movement, respiratory rate, heart rate, electroencephalogram signals.

18. The apparatus of claim 15, further comprising a battery.

19. The apparatus of claim 18, further comprising a charging circuit and a charging antenna configured to recharge the battery from power inductively received by the charging antenna.

20. The apparatus of claim 15, further comprising a charging antenna configured to power the controller and the thermoelectric temperature regulators when power is received by the charging antenna.

21. The apparatus of claim 15, further comprising a wireless communications circuit configured to wirelessly transmit and receive to and from the controller.

22. The apparatus of claim 15, wherein the controller is configured to apply energy in a pulsatile manner so that the thermoelectric temperature regulators apply pulsed cooling to the forehead.

23. The apparatus of claim 23, wherein the energy is applied in pulses having a pulse duration of less than 360 seconds.

24. The apparatus of claim 15, further comprising a removable cover having a thermally transmissive surface configured to be positioned over the skin-facing surface.
